(19) United States
(12) Patent Application Publication
(10) Pub. No.: US 2015/0250416 A1
(43) Pub. Date: Sep. 10, 2015

(54) APPARATUS AND METHOD TO ASSESS WOUND HEALING

(71) Applicant: VASAMED, INC., Eden Prairie, MN (US)

(72) Inventors: Paulita M. LaPlante, Inver Grove Heights, MN (US); Daniel J. Bartnik, Eden Prairie, MN (US); Chuck Yetter, Eden Prairie, MN (US)

(21) Appl. No.: 14/431,556

(22) PCT Filed: Oct. 4, 2013
(86) PCT No.: PCT/US13/63502

§ 371 Date: Mar. 26, 2015

Related U.S. Application Data
(60) Provisional application No. 61/710,402, filed on Oct. 5, 2012.

(51) Int. Cl. A61B 5/00

(52) U.S. Cl. CPC A61B 5/445; A61B 5/743

(57) ABSTRACT

A device and method for measuring wounds is provided. The device includes a guide having cross-hairs that are positioned on a wound. A computer generated caliper guide having a known dimension is placed on one of the cross-hairs. The outer perimeter or inner perimeter of the a computer-generated image of the wound is marked to form a polygonal shape. From this shape, the surface area of the wound including its volume may be calculated by computer means.

### FIELD OF THE INVENTION

The invention relates to the field of wound assessment and measurement.

### BACKGROUND OF THE INVENTION

Other than the PAD-IQ itself, an apparatus for non-invasive measurement of perfusion pressures and pulse volume recording waveforms disclosed in U.S. Pat. Nos. 7,736,311 and 8,133,177 and co-pending application Ser. No. 12/321,813 the entireties of which are hereby incorporated by reference, no special camera or imaging equipment is needed. An image can be pulled into PAD-IQ from any standard camera via one of the PAD-IQ USB ports. The PAD-IQ software will recognize the camera on subsequent uses following the first time a user downloads pictures from their camera to the PAD-IQ.

### BRIEF DESCRIPTION OF THE DRAWINGS

FIG. 1 is a view of a wound being measured with the guide in accordance with one embodiment of the invention.

FIG. 2 is a three-dimensional illustration of a wound to be measured with the device in accordance with an embodiment of the invention.

FIG. 3 is a three-dimensional illustration of a wound to be measured with the device in accordance with an embodiment of the invention.

FIG. 4 is a three-dimensional illustration of a wound to be measured with the device in accordance with an embodiment of the invention.

### DETAILED DESCRIPTION OF THE INVENTION

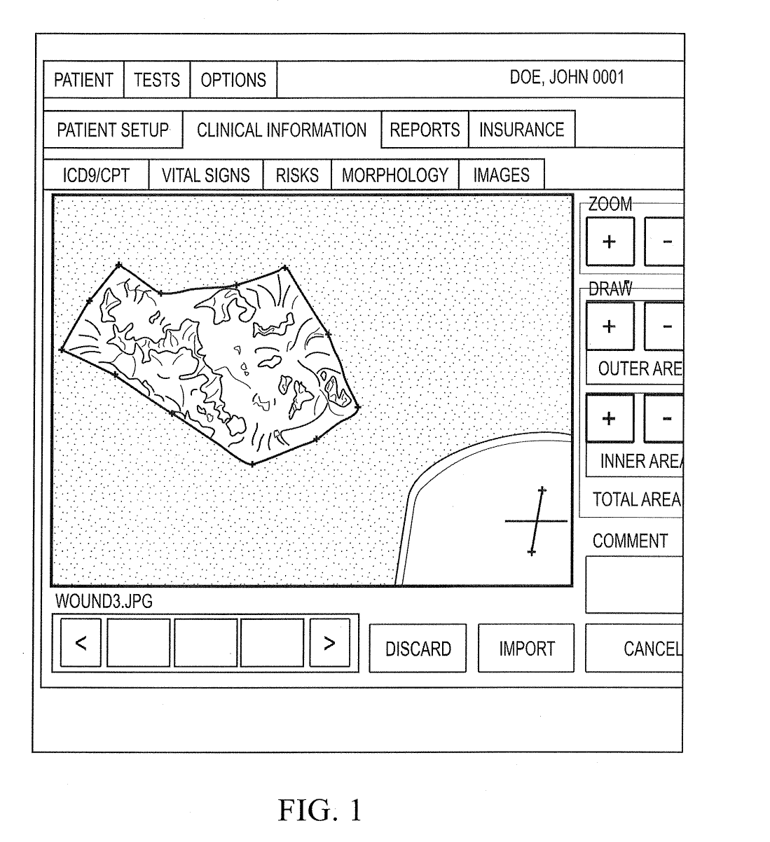

The image goes to the PAD-IQ wound image file wherein it can be assessed. Two surface areas can be calculated and an inner and outer margin could be described. The “SmartGuide” depicted in FIG. 1 is used to enable a caliper tool. The green dotted line on the gray cross hairs of the SmartGuide provides the user with a known distance of 1 cm. As soon as the user brings in an image, zooms in and centers it so that the SmartGuide cross-hairs and the wound are both visible, “Scale” is selected. A green dotted line appears and the user adjusts it to either the horizontal or vertical line of the cross hairs. After the scale is set, the user then touches the “+ icon under “Draw” and then touches several points around the wound perimeters of interest (inner margin and/or outer margin). If a mistake is made regarding any particular touch point, the user can touch the “-” icon and the incorrect point is deleted. Then the user touches the ⇄ icon. This permits the user to customize the polygon shape around the selected area. The Surface Area (SA) is then auto-calculated. The images and SA calculations are saved and can be archived and retrieved for review and monitored over time. Optionally, graphical charts (bar or line) may also be displayed.

By placing the caliper tool on both the horizontal and vertical line of the cross hairs, information is provided about the plane in which the wound lies. This is important to improved accuracy of area calculation. It also assists in obtaining information about distance of the camera from the wound. Reflective means are also included on the SmartGuide to assist in obtaining information about distance of the camera from the wound.

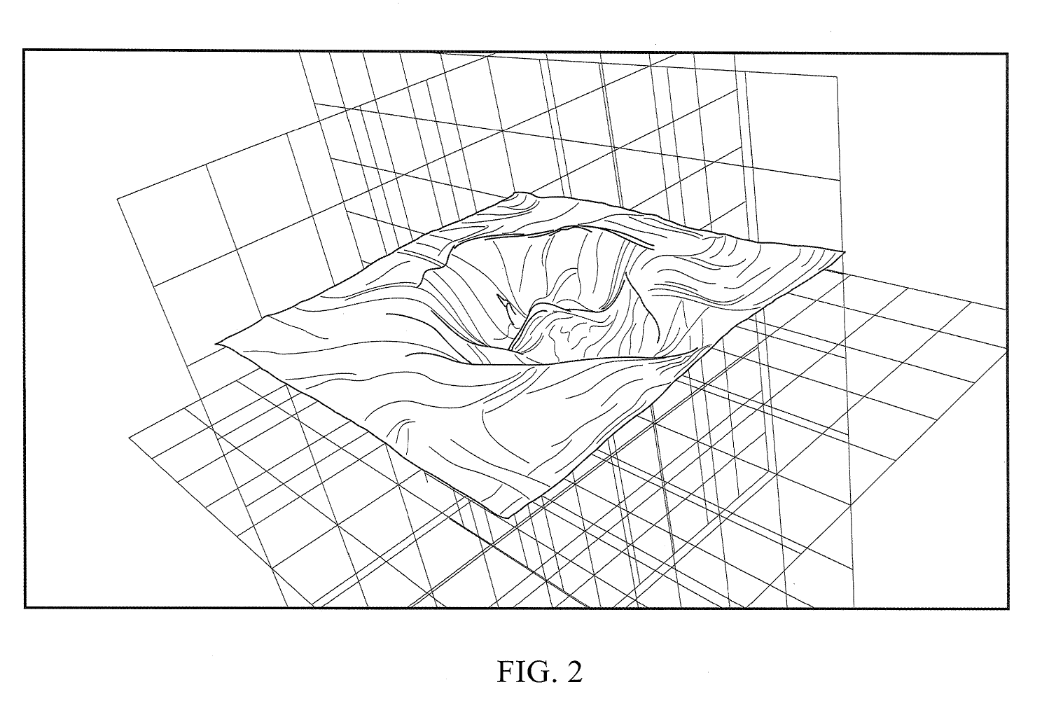

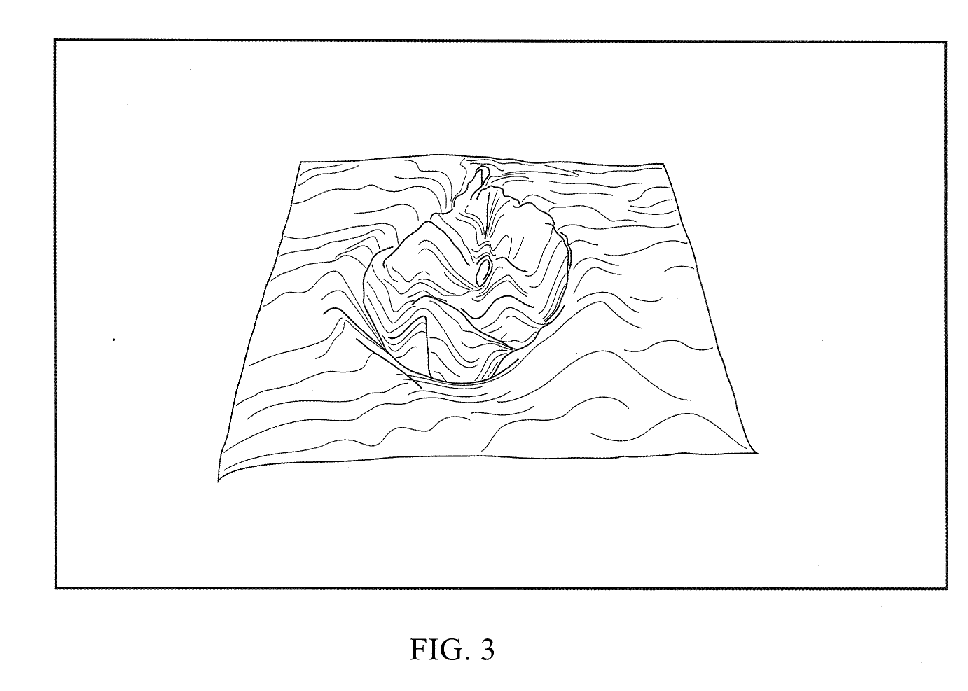

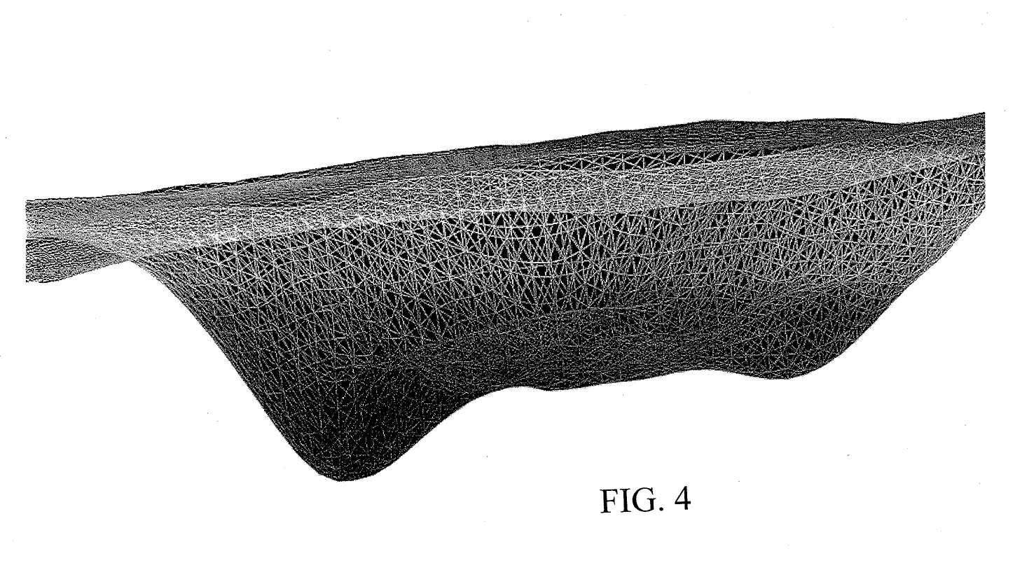

The known colors on the SmartGuide may also be used to calibrate the colors in the photo of the wound. The colors of the wound itself can be used to create Skin Color Recognition (SCR) zones. SCR coupled with known plane and surface area permits a construct of 3-D models of the wound that allows the user to estimate wound volume. Because lower extremity wounds in Diabetic patients are very deep, it is important to track the volume as well as the surface area of the wound over time in order to evaluate the success of various therapies used to heal wounds. Exemplary 3-D models are submitted herewith as FIGS. 2, 3 and 4.

Although the present invention has been described with reference to preferred embodiments, workers skilled in the art will recognize that changes may be made in form and detail without departing from the spirit and scope of the invention.

We claim:

1. A method for measuring wounds comprising:
providing a device including a crosshair having an X-axis and a Y-axis;
placing said device on a wound of a patient;
providing a computer-generated image of a wound on a screen;
placing the crosshair on the computer-generated image of the wound;
providing a computer-generated caliper guide having a known distance;
placing said computer-generated guide on either the X-axis or the Y-axis of said crosshair;
marking an outer perimeter or an inner perimeter on the computer-generated image of the wound to generate a polygonal shape;
analyzing said polygonal shape by computer means; and
calculating a surface area and volume of the wound.
